(19) United States
(12) Patent Application Publication
(10) Pub. No.: US 2022/0059984 A1
(43) Pub. Date: Feb. 24, 2022

(54) DRIVER CIRCUIT, METHOD FOR LASER ACTUATION, AND OPHTHALMOLOGICAL LASER TREATMENT DEVICE

(71) Applicant: MERIDIAN AG, Thun (CH)

(72) Inventor: Michael STETTER, Lüsslingen (CH)

(73) Assignee: MERIDIAN AG, Thun (CH)

(21) Appl. No.: 17/425,120

(22) PCT Filed: Jan. 22, 2020
(86) PCT No.: PCT/EP2020/051509

§ 371 Date: Jul. 22, 2021

(30) Foreign Application Priority Data
Jan. 22, 2019 (CH) 00070/19

(51) Int. Cl. H01S 5/026; H01S 5/042; H05B 47/105

(52) U.S. Cl. CPC H01S 5/0261; H05B 47/105; H01S 5/0428

(57) ABSTRACT

The invention relates to a driver circuit (1) for generating a current flow through a light source (5), in particular a laser diode (LD), to a method for operating the driver circuit (1) and to an ophthalmological laser treatment device comprising such a driver circuit (1). A voltage source (3), the light source (5), a series resistor and a switch are arranged in an electric circuit (2). To generate a series resistance which can be controlled or regulated in discrete stages for the light source (5), the electric circuit (2) is branched into at least two parallel branch circuits (21, . . . , 2N), and there is at least one connectable series resistor (R1, . . . , RN) in each branch circuit (21, . . . , 2N).

### TECHNICAL FIELD

The invention relates to the field of the current regulation for the actuation of laser light sources. It is based on a driver circuit, in particular driver electronics, and a method according to the preamble of the corresponding independent patent claims. The present invention further relates to an ophthalmological laser treatment device comprising a driver circuit for a laser actuation according to the invention.

### PRIOR ART

The starting point of the invention is WO 2013/046285 A1. The document discloses a driver circuit for a light source (e.g., light-emitting diode LED), which makes it possible to compensate for an environmentally variable voltage drop by means of the LED. For this purpose, a second regulated shunt current driver path is provided in parallel to the first current driver path, the electrical resistance of which is regulated in such a way that the heat dissipation in the shunt resistor counteracts the variable current by means of the LED, thereby stabilizing the light output of the LED.

DE 10 2016 212 928 A1 discloses a method for generating a laser pulse of an excitation laser in response to a trigger time of a trigger signal. In this method, the driver actuation signal is generated in consideration of the time interval between the current trigger time and the previous trigger time.

DE 103 93 192 T5 discloses a circuit for the joint supply of power to a plurality of light-emitting diodes (LEDs), wherein the LEDS are arranged in parallel current paths and each LED is assigned its own current regulator for the regulation of its light output.

DE 20 2010 017 580 U1 discloses a circuit arrangement for reducing the power dissipation of linear current drivers for LEDs. A control variable for the LED supply voltage is determined from the power dissipation of the driver stage and routed as a control signal to a control network for adjusting the LED supply voltage. The driver circuit is independent of the type of LED and is suitable for a static or a dynamic actuation.

### DESCRIPTION OF THE INVENTION

It is an object of the present invention to provide an improved driver circuit and laser actuation method, which allow for a simplified regulation of the light output. Another object of the present invention is to propose an ophthalmological laser treatment device that allows for improved ophthalmological treatments due to precisely shaped laser pulses. According to the invention, these objects are achieved by the subject matters of the independent claims. Further features, in particular in subclaims or the combination of subclaims or the combination of embodiments, are optional and therefore designated with the terms “in particular,” “preferably and the like,” and are therefore not essential to the invention but rather serve to achieve further advantages or effects.

In many laser processing applications for materials and tissue, especially in ophthalmological laser treatments, it is necessary to generate laser beam pulses with certain temporal characteristics in order to optimize the efficiency of the processing or the treatment and to minimize side effects: this concerns the rise, progression and drop of the pulse. This is especially true when using reproducible pulses (single pulses or pulse trains) of a few milliseconds for ophthalmological laser treatments. For example, pulse lengths of a few microseconds are required for the Selective Retinal Laser Therapy (SRT). The pulses must have a controlled reproducible rise and progression for treatments at the process threshold. The present invention describes a driver circuit that is suitable for this purpose, a method for regulating the output power of laser diodes or diode-pumped lasers, and an ophthalmological laser treatment device.

In a first aspect, the invention comprises a driver circuit for generating a current flow through a light source, in particular a laser diode, wherein a voltage source, the light source, a series resistor and a switch are arranged in an electric circuit, wherein, in order to generate a series resistor for the light source which can be controlled in discrete stages, the electric circuit is branched into at least two parallel branch circuits and at least one connectable series resistor is present in each branch circuit. By regulating the light source in discrete stages by connecting or disconnecting discrete parallel series resistors, analog or digital controllers for the series resistor can be avoided and an overall simple and fast control of the driver circuit that is basically stable with regard to external influences can be achieved. The light source is, in particular, a laser diode or a laser diode-pumped or light diode-pumped laser resonator.

Various embodiments of the invention relate, among other things, to the use of a larger number N of branch circuits, e.g., N>=2 or N>=5 or N>=10; the selection of the same or different series resistors; the actuation, i.e., the switching on or off of one switch each per branch circuit by a microcontroller or programmable logic or digital electronics; and a voltage source with a variable output voltage.

In a second aspect, the invention consists of a method for operating the driver circuit disclosed herein, wherein the series resistor is regulated in discrete stages by switching at least one of the switches on or off.

Various embodiments in this regard relate to, among other things, a timed sequence of switch configurations which is implemented in such a way that a predetermined current-time curve or light output-time curve is realized for the light source; a first rapid timed sequence of switch configurations is implemented in such a way that short rise times of the light output, e.g., in the range of 0.1-100 microseconds, are achieved at the beginning of a current pulse; a second subsequent timed sequence of switch configurations is implemented in such a way that the light output of the light source is regulated in dependence of a current measurement or light output measurement; and a third slow sequence of switch configurations is implemented in such a way that, in the course of a current pulse, fluctuations of the light output of the light source, due to thermal effects, are compensated.

In another preferred embodiment of this second aspect of the present invention, the method is used to control a laser or a laser diode in an ophthalmological laser treatment, in particular in a Selective Retina Therapy (SRT) or in a micro pulse/“sub-threshold” laser therapy. This can improve the treatment outcomes.

In a third aspect, the invention relates to an ophthalmological laser treatment device, in particular for Selective Retina Therapy (SRT) or for the micro pulse/“sub-threshold” laser method, comprising a laser or a laser diode and a driver circuit for generating a current flow through the laser or the laser diode, wherein, in a circuit of the driver circuit, a voltage source, the laser or the laser diode, a series resistor and a switch are arranged, characterized in that, in order to generate a series resistor for the laser or the laser diode which can be controlled or regulated in discrete stages, the current circuit is branched into at least two parallel branch circuits, and at least one connectable series resistor is present in each branch circuit. This makes it possible to realize ophthalmological laser treatment devices in which the control of the laser or the laser diode can be performed in discrete stages by connecting or disconnecting discrete parallel series resistors and without requiring analog or digital controllers for the series resistor. In addition, this makes it possible to realize ophthalmological laser treatment devices with an overall simple and fast control of the generated light that is basically stable with regard to external influences. Compared to devices known from the prior art, such ophthalmological laser treatment devices have the advantage that laser pulses with a predetermined shape can be generated in a precise and easy manner. In particular, the ability to form precisely shaped pulses for all pulses of pulse trains is very important in the field of ophthalmological treatments.

Various embodiments of the invention relate, among other things, to the use of a larger number N of branch circuits in the driver circuit, e.g., N>=2 or N>=5 or N>=10; the selection of the same or different series resistors; the actuation, i.e., the switching on or off, of one switch per branch circuit by a microcontroller or programmable logic or digital electronics; and a voltage source with a variable output voltage.

The use of green or yellow laser light is particularly important in ophthalmology. Frequency-doubled, optically pumped laser systems (DPSSL, OPSL, etc.) are commonly used to generate green or yellow laser light. Certain treatment methods, e.g., the Selective Retina Therapy (SRT) or micro pulse/“sub-threshold” laser therapy, require pulses in the microsecond range. In order to be able to generate such fast pulses with an ophthalmological laser treatment device according to the invention, it is particularly advantageous if the current of the laser diode or the laser can be increased to a certain value at a certain point in time prior to the actually desired laser pulse so that the laser treatment device is then able to quickly deliver the actually desired pulse.

It is also advantageous if the laser treatment device is configured to allow for a certain timed sequence of the switch combinations so that light pulses with a specific pulse shape can be generated. It is advantageous in ophthalmology to generate pulse shapes that are as “rectangular” as possible, i.e., pulses with a very short power-on time followed by a constant output. In contrast to known devices, the ophthalmological laser treatment device according to the invention requires a single driver circuit for the generation of very short but also long laser pulses. This makes it possible to use different treatment methods known in ophthalmology, such as the Selective Retina Therapy (SRT), micro pulse/“sub-threshold” laser methods or traditional laser photocoagulation with a single laser treatment device. Previously, this required multiple ophthalmological devices such as a CW cavity with driver and also a Q-switched cavity with its own driver, as well as an optical switch or even two completely separate devices. Thus, a much simpler laser treatment device than before can be provided.

Furthermore, due to the capability of forming nearly rectangular light pulses, the same energy can be deposited in the treatment site of the eye at the same time but with a lower absolute light output. This is particularly advantageous in ophthalmology to achieve the most efficient treatment with the least possible damage to the tissue.

Thanks to an ophthalmological laser treatment device according to the invention, the efficiency achieved in the generation of the laser light (conversion of electrical energy into laser light energy) is good as well. This is advantageous because it allows a laser treatment device to be constructed without a fan, which makes it much easier to use, for example, in operating rooms.

It is important to note that the “Selective Retina Therapy” (SRT) requires laser pulses of a few microseconds and below. Until now, complex and expensive Q-switched laser systems have been used for this purpose. With the help of the described ophthalmological laser treatment device, a simpler CW laser system (e.g., DPSSL, OPSL, etc.) can be regulated in such a way that laser pulses with the required pulse duration and laser output can be generated. This allows for the manufacture of a much cheaper SRT treatment device than previously possible with the additional possibility to generate longer laser pulses for other ophthalmological treatment methods (e.g., micro pulse/“sub-threshold” laser methods and/or traditional laser photocoagulation).

It is particularly advantageous if the ophthalmological laser treatment device according to the invention is configured such that laser pulses with a length in the range of 0.5-50 microseconds, preferably 1-10 microseconds, can be generated and/or that laser pulses can be generated with a fast reproducible rise in the range of 0.1-5 microseconds, preferably 0.5-2 microseconds.

Further embodiments, advantages and applications of the invention can be obtained from the dependent claims, from the combinations of claims and from the descriptions and figures below.

### BRIEF DESCRIPTION OF THE DRAWINGS

The figures schematically show

FIG. 1 a simple self-stabilizing driver circuit according to the prior art;

FIG. 2 a first embodiment of a driver circuit according to the invention;

FIG. 3 a second embodiment of a driver circuit with a microcontroller according to the invention;

FIG. 4,5 third embodiments of a driver circuit with a microcontroller and feedback according to the invention;

FIG. 6 a current-light output diagram of a light-emitting diode (LED) or laser diode (LD);

FIG. 7 a substantially ideal time response of the current and the light output of an LED or LD; and

FIG. 8 a real-time behavior of the current and the light output of a laser diode.

In the figures, identical or functionally identical parts are provided with the same reference signs.

### WAYS IN WHICH TO REALIZE THE INVENTION

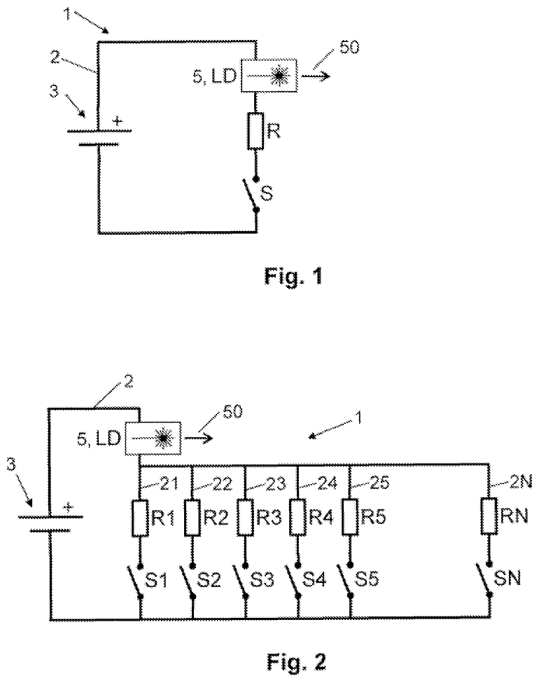

FIG. 1 shows the principle, known per se, of a driver circuit 1 for generating a light emission 50 by means of a circuit 2 with a voltage source 3, a light source 5, in particular a laser diode LD, a series resistor R and a switch S. The circuit is self-stabilizing and does not require any active components or controllers. The strength of the current can be easily determined with the voltage of the voltage source 3 and the resistance value R. By opening and closing the circuit 2 with the switch S (e.g., MOSFET) the current and thus the light can be pulsed. The disadvantage here is that the current setpoint can only be varied by changing the voltage of the voltage source or by changing the resistance value R, which requires substantial technical effort. Furthermore, a high voltage of the voltage source 3 may, under certain circumstances, lead to a high power dissipation in the resistor R.

Conventional driver circuits are based, for example, on analog electronics: In principle, the current can be regulated, for example, by means of operational amplifiers and corresponding power semiconductors. The disadvantage here is that controllers can be fundamentally unstable, and the setting of the controller parameters and the selection of the components is not trivial, especially in the case of variable control setpoints. Digital electronics or digital switching controllers are widespread as well: Current switching controllers generally offer a good energy efficiency. The disadvantage here as well is that complex controller algorithms are required. In addition, very fast microcontrollers or e.g., “field-programmable gate arrays” (FPGAs) are required.

One object of the invention is a driver circuit 1 and a method for operating the driver circuit 1 according to the independent claims. Embodiments in this regard are provided below. It is also an object of the present invention to provide an ophthalmological laser treatment device comprising a driver circuit according to the invention.

FIG. 2 shows an embodiment of a driver circuit 1 with a circuit 2 that is branched into N parallel branch circuits 21, . . . , 2N, each of which has a resistor R1, . . . , RN, also called a series resistor, and a switch S1, . . . , SN, in particular an on/off switch S1, . . . , SN, in series.

The principle presented here allows for the development of simple electronic hardware and software for generating light pulses in laser diodes and laser systems, especially in ophthalmological laser treatment devices. The principle is based on the “voltage source with series resistor” setup described in FIG. 1. However, the series resistor is newly implemented as a combination of a plurality of individually connectable resistors R1, . . . , RN.

The total resistance R can be calculated, depending on the switch positions. In particular, R⁻¹=(R1ₐₙ⁻¹+ . . . +Rnₐₙ⁻¹), where index “an” denotes in each case the resistor R1ₐₙ, . . . , Rnₐₙ arranged in a connected branch circuit and the running index is n<=N.

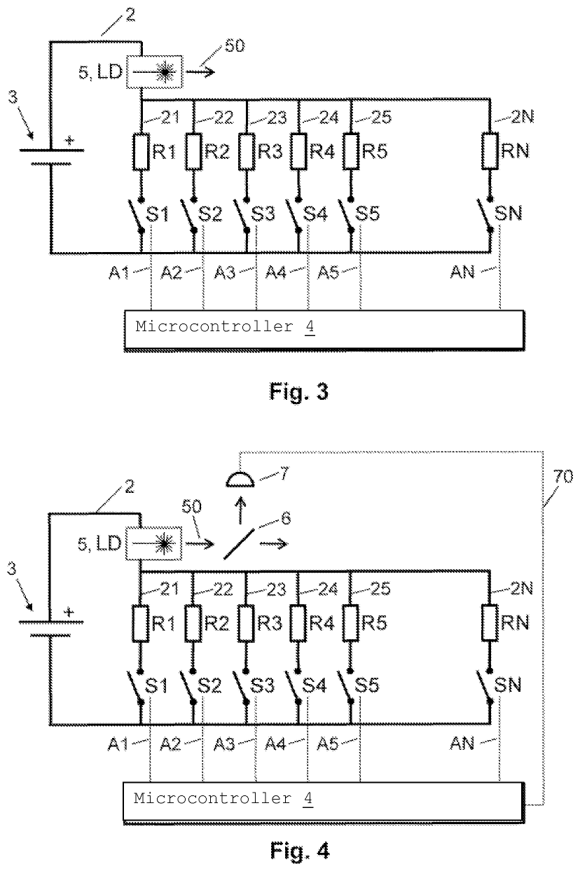

In FIGS. 3-5, the switches S1 to SN can be actuated by digital electronics or a microcontroller 4. The times at which the switches S1, . . . , SN change their state, i.e., at which a new switch combination is set, is variable. According to the invention, this can be done very slowly but also very quickly.

According to the embodiment in FIG. 3, the microcontroller 4, or alternatively digital electronics, is connected to each switch S1, . . . , SN via a respective actuation path (A1, . . . , AN). According to the embodiment in FIG. 4, a light sensor 6, 7, in particular comprising a beam splitter 6 and a photodetector 7, is additionally provided for the light source 5 for measuring the light output of the light source 5, which is connected to the microcontroller 4, or alternatively to the digital electronics, via a feedback path 70 in order to regulate the light output. According to the embodiment example in FIG. 5, a current sensor 8 is present in the electric circuit 2 for measuring the current that is present due to the light source 5 and is connected to the microcontroller 4 or alternatively to the digital electronics via a feedback path 80 in order to regulate the current intensity. The embodiments of FIGS. 4 and 5 can also be implemented in combination with each other, i.e., a feedback is used for both the current intensity and the light output.

The term microcontroller 4 is also intended to include programmable logic. In particular, the microcontroller 4 comprises software that is implemented in the driver circuit 1 and that is part of the driver circuit or programmed to execute the method.

In further embodiments, some or all of the series resistors R1, . . . , RN have the same resistance value or some or all of the series resistors R1, . . . , RN have different resistance values, in particular doubled resistance values in each case, according to the formula RN=2^{(N-1)}R1, wherein N=the number of branch circuits or the number of series resistors R1, . . . , RN.

In embodiments, doubled resistance values may be selected with respect to the nearest resistor in each case, for example R1=1Ω, R2=2Ω, R3=4Ω, R4=8Ω, R5=16Ω, . . . , RN=2^{N}Ω, or more generally expressed: R2=2*R1, R3=4*R1, R4=8*R1, R5=16*R1, . . . , RN=2^{(N-1)}*R1.

In other embodiments that are not shown, the voltage source 3 may include means for varying its output voltage and/or the light source 5 may be a laser diode LD or a laser diode-pumped or light diode (LED)-pumped laser system, in particular a diode-pumped solid-state laser DPSSL or a coherent optically pumped semiconductor laser OPSL.

The method for operating the driver circuit 1 consists in that a voltage source 3, a light source 5, a series resistor and a switch are arranged in a circuit 2, wherein the circuit 2 is branched into at least two parallel branch circuits 21, . . . , 2N, in that each branch circuit 21, . . . , 2N comprises a series resistor R1, . . . , RN and a switch S1, . . . , SN for connecting or disconnecting the series resistor R1, . . . , RN, wherein the series resistor is thus controlled or regulated in discrete stages by switching on or off at least one of the switches S1, . . . , SN.

Preferably, N branch circuits 21, . . . , 2N are present, wherein N=a natural number greater than or equal to 2. Furthermore, 2^{N} switch configurations are to be present due to the turning on and turning off of the N switches S₁, . . . , S_{N}, wherein the series resistor is controlled or regulated in stages by a timed sequence of switch configurations.

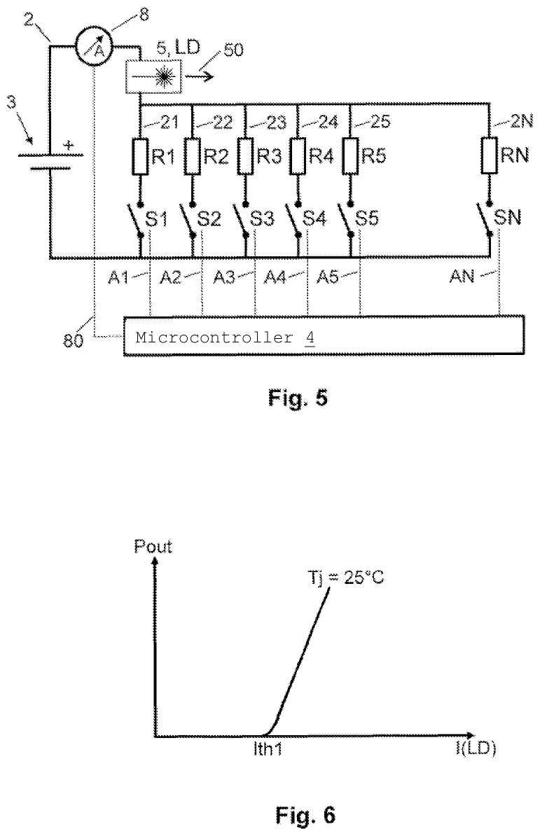

FIG. 6 shows a typical characteristic curve of a laser diode (LD) or light-emitting diode (LED) for the emitted light output Pₒᵤₜ [in watts] as a function of the current I [in amperes], shown here for a junction temperature Tj of 25° C. Light emission takes place only after the current I has exceeded a certain threshold value Iₜₕ₁.

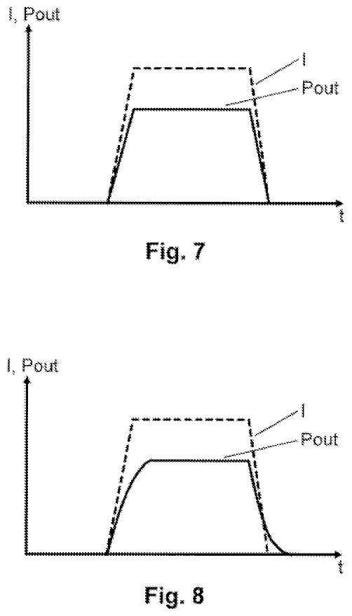

FIG. 7 shows a basically ideal behavior of the characteristic curve of the current and the light output of a laser diode (LD) or light-emitting diode (LED) over time. The light output emitted by laser diodes or light-emitting diodes and laser systems (DPSSL, OPSL, etc.) in the time range under consideration here is often such that the output light intensity Pₒᵤₜ of the electric current intensity I through the laser diode occurs very quickly.

FIG. 8 shows an example in which the laser diode or the laser system does not have a directly proportional relationship between the output power and the current in the time range under consideration. The oscillation starts and ends only after a certain delay. An overshooting can also occur under certain circumstances.

Real laser diodes and real voltage sources thus have non-ideal properties. Mainly, the parasitic series inductance of the laser diode and the supply line have an impact. Furthermore, the ohmic and inductive internal resistance, a limited capacitance and non-ideal load-regulating properties of the voltage source result in a short-time change in the voltage when the switch is turned on.

Such non-idealities of laser diodes or laser systems can be reduced or largely compensated for by the driver circuit 1 and the method according to the invention, and likewise by the disclosed embodiments.

In embodiments, a timed sequence of switch configurations is implemented in such a way that a predetermined current-time curve or light output-time curve Pₒᵤₜ(t) is realized for the light source 5, in particular, in order to at least partially compensate for a non-ideal behavior of the light source 5 and/or of the voltage source 3 and/or of supply lines when the light source 5 is switched on. This also allows for the possibility of pulse design or shape engineering, i.e., the possibility of shaping the shape of the laser (Pₒᵤₜ(t)) in a manner that is optimal for the application, in particular the ophthalmological treatment.

In particular, a first timed sequence of the switch configurations can be executed in such a way that, in order to generate short rise times of the light output, for example in the range of 0.1-100 microseconds, a very small series resistance is set at the beginning of a current pulse, and this series resistance is increased in phases by changing the switch configuration, in particular after 1 microsecond in each case. The first timed sequence is thus preferably executed on a very short time scale, in particular microseconds or sub-microseconds such as 1 ns-1000 ns or preferably 100 ns-1000 ns.

Alternatively or in addition, a second timed sequence of switch configurations may be executed such that, after the first timed sequence, as time progresses, the light output of the light source 5 is regulated as a function of a current measurement in the electric circuit 2 or a light output measurement of the current source 5 by varying the series resistance in stages. The second timed sequence is preferably executed on a slower time scale compared to the first timed sequence, for example in the range of 1-1000 microseconds or preferably 5-500 microseconds or particularly preferably 10-200 microseconds.

Alternatively or in addition, a third timed sequence of switch configurations can be executed in such a way that, in the course of a current pulse, fluctuations in the light output of the light source 5 due to thermal effects, in particular in the voltage source 3 and/or in the series resistors R1, . . . , RN, are compensated for by discrete or phased changes in the series resistor. The third timed sequence is preferably executed on an even slower time scale, e.g., in the range of seconds or minutes.

In further embodiments, the voltage source 3 is controlled or regulated in such a way that, at the beginning of a current pulse, a higher voltage is selected and thereafter gradually reduced, in particular in order to realize an approximately rectangular waveform of the light output Pₒᵤₜ(t) of the light source 5 as a function of time t and/or to keep the power dissipation in the resistors small.

In further embodiments of the driver circuit and the method, the oscillation of the light source, in particular a laser or laser diode-pumped laser system, can be improved by bringing the pump current for the laser 5, either continuously or during a lead time before the desired laser light output, in particular for the laser diode 5 or pump laser diode 5, to a value just below the threshold current Iₜₕ₁ by switching the switches S1, . . . , SN. The lead time can be selected, for example, in the range of 1 microsecond-100 microseconds.

### REFERENCE LIST

1 Driver circuit, laser driver circuit, LED driver circuit
2 Electric circuit
21, . . . 2N Parallel current paths, parallel branch circuits
3 Voltage source
4 Microcontroller, digital electronics
5 Light source, laser, laser system, laser diode, laser diode pumped laser system, diode pumped solid state laser (DPSSL), coherent optically pumped semiconductor laser (OPSL)
50 Emitted light
6, 7 Light sensor
6 Beam splitter
7 Photodetector, light output measurement
70 Feedback path for the light output control
8 Current sensor, current measurement
80 Feedback path for the current intensity control
9 Current intensity [amperes] through the laser diode
10 Light output [watts] of the laser diode
LD Laser diode
R Electrical resistance
R1, . . . , RN Electrical resistors 1, 2, . . . , N in parallel current paths
S Switch, MOSFET
S1, . . . , SN Switches in parallel current paths, MOSFETS
N Number of parallel current paths
A1, . . . , AN Actuation paths 1, 2, . . . , N from the microcontroller to the 1st, 2nd, . . . , Nth switch
I Current [amperes] through the light source
I(LD) Current flow through the laser diode
Iₜₕ₁ Current threshold of the laser diode
Pₒᵤₜ Light output [watts] of the light source
Tⱼ Temperature of the laser diode, junction temperature
t Time [second]

1. Driver circuit (1) for generating a current flow through a light source (5), in particular a laser diode (LD) or a light-emitting diode (LED), wherein a voltage source (3), the light source (5), a series resistor and a switch are arranged in an electric circuit (2), characterized in that, in order to generate a series resistor for the light source (5) that can be controlled or regulated in discrete stages, the circuit (2) is branched into at least two parallel branch circuits (21, . . . , 2N), and, in each branch circuit (21, . . . , 2N), there is at least one connectable series resistor (R1, . . . , RN).

2. Driver circuit (1) according to claim 1, characterized in that at least one switch (S1, SN) is provided in each branch circuit (21, . . . , 2N) for connecting or disconnecting the series resistor (R1, . . . , RN).

3. Driver circuit (1) according to claim 1, characterized in that
a) series resistors (R1, . . . , RN) with the same resistance value are present and/or
b) series resistors (R1, . . . , RN) with different resistance values, in particular in a series with doubled resistance values (RN=2^{(N-1)}R1), are present.

4. Driver circuit (1) according to claim 2, characterized in that
a) in each branch circuit (21, . . . , 2N), the switch (S1, SN) is arranged in series with the series resistor (R1, . . . , RN) and/or
b) the switches (S1, . . . , SN) are MOSFET switches.

5. Driver circuit (1) according to claim 2, characterized in that digital electronics or a microcontroller (4) is provided for controlling the switches (S1, . . . , SN), in particular the digital electronics or the microcontroller (4) is connected to each switch (S1, . . . , SN) via a respective actuation path (A1, . . . , AN).

6. Driver circuit (1) according to claim 5, characterized in that
a) a current sensor (8) for measuring the current through the light source (5) is present in the electric circuit (2) and connected to the digital electronics or the microcontroller (4) via a feedback path (80) for the current intensity control and/or
b) a light sensor (6, 7) for measuring the light output (Pₒᵤₜ) of the light source (5) is provided for the light source (5) and is connected to the digital electronics or the microcontroller (4) via a feedback path (70) for the light output control.

7. Driver circuit (1) according to claim 1, characterized in that
a) the voltage source (3) comprises means for varying its output voltage and/or
b) the light source (5) is a laser or a laser diode (LD) or a laser diode-pumped or LED-pumped laser system, in particular a diode-pumped solid-state laser (DPSSL) or a coherently optically pumped semiconductor laser (OPSL) and/or
c) the driver circuit (1) comprises means for carrying out a method for operating the driver circuit (1), wherein in each branch circuit (21, . . . , 2N), a series resistor (R1, . . . , RN) and a switch (S1, SN) for connecting or disconnecting the series resistor (R1, . . . , RN) are present, characterized in that the series resistor is controlled or regulated in discrete stages by switching on or off at least one of the switches (S1, . . . , SN).

8. Method for operating a driver circuit (1) according to claim 1, wherein a voltage source (3), a light source (5), a series resistor and a switch are arranged in an electric circuit (2), wherein the electric circuit (2) is branched into at least two parallel branch circuits (21, . . . , 2N); in each branch circuit (21, . . . , 2N), a series resistor (R1, . . . , RN) and a switch (S1, . . . , SN) for connecting or disconnecting the series resistor (R1, . . . , RN) are present, characterized in that the series resistor is controlled or regulated in discrete stages by switching on or off at least one of the switches (S1, . . . , SN).

9. Method according to claim 8, characterized in that N branch circuits (21, . . . , 2N) are present where N=a natural number greater than or equal to 2, in that 2^{N} switch configurations are present due to the on or off position of the N switches (S1, . . . , SN) and, in particular, are connectable, and in that the series resistor is controlled or regulated in stages by a timed sequence of the switch configurations.

10. Method according to claim 9, characterized in that a timed sequence of switch configurations is executed in such a way that a predetermined current-time curve or light output-time curve (Pₒᵤₜ(t)) is realized for the light source (5).

11. Method according to claim 9, characterized in that the timed sequence of the switch configurations is executed in such a way that (i) a non-ideal behavior of the light source (5) and/or of the voltage source (3) and/or of the supply lines is at least partially compensated for when the light source (5) is switched on and/or (ii) that, for an improved start-up of the laser system (5), the current for pumping the laser system (5) is controlled or regulated continuously or during a lead time before a desired laser light output (50), in particular selected in the range of 1 microsecond-100 microseconds, to a value just below the threshold current (Iₜₕ₁).

12. Method according to claim 10, characterized in that
a) a first timed sequence of the switch configurations is executed in such a way that, in order to generate short rise times) of the light output, e.g., in the range of 0.1-100 microseconds, a very small series resistance is set at the beginning of a current pulse and that this series resistance is increased stepwise by changing the switch configuration, in particular in each case after a time interval in the range of 1 microsecond or in the range of 1 ns-1000 ns and/or
b) a second timed sequence of the switch configurations, in particular on a time scale in the range of 1-1000 microseconds, is executed in such a way that, as time progresses, in particular after the first timed sequence, the light output (Pₒᵤₜ) of the light source (5) is controlled on the basis of a current measurement in the electric circuit (2) or a light output measurement of the current source (5) by gradually changing the series resistor and/or
c) a third timed sequence of the switch configurations, in particular on a time scale in the range of seconds or minutes, is executed in such a way that, in the course of a current pulse, fluctuations of the light output (Pₒᵤₜ) of the light source (5) due to thermal effects, in particular in the voltage source (3) and/or in the series resistors (R1, . . . , RN), are compensated for by gradually changing the series resistor.

13. Method according to claim 8, characterized in that the voltage source (3) is controlled or regulated in such a way that, at the beginning of a current pulse, a higher voltage is selected and then gradually decreased, in particular in order to realize an approximately rectangular curve shape of the light output (Pₒᵤₜ(t)) of the light source (5) as a function of time (t) and/or in order to keep the power dissipation in the resistors small.

14. Method according to claim 9, characterized in that the light source is a laser system (5), e.g., a laser diode (5) or a diode-pumped laser (5), and in that the timed sequence of the switch configurations is executed in such a way that the laser pulse shape and the laser pulse frequency are adapted or optimized for ophthalmological applications, in particular for Selective Retina Laser Therapy.

15. Method according to claim 14, characterized in that the laser pulses are generated with a length in the range of 0.5-50 microseconds, preferably 1-10 microseconds and/or the laser pulses are generated with a fast reproducible rise in the range of 0.1-5 microseconds, preferably 0.5-2 microseconds.

16. Method according to claim 8, wherein the method is used to control a laser or a laser diode in an ophthalmological laser treatment, in particular in a Selective Retina Therapy (SRT) or in a micro pulse/“sub-threshold” laser method.

17. Ophthalmological laser treatment device, in particular for the Selective Retina Therapy (SRT) or for the micro pulse/“sub-threshold” laser therapy, comprising a laser or a laser diode (LD) and a driver circuit (1) for generating a current flow through the laser or the laser diode (LD), wherein, in an electric circuit (2) of the driver circuit, a voltage source (3), the laser or the laser diode (LD), a series resistor and a switch are arranged, characterized in that, in order to generate a series resistor for the laser or the laser diode (LD) which can be controlled or regulated in discrete stages, the circuit (2) is branched into at least two parallel branch circuits (21, . . . , 2N) and at least one connectable series resistor (R1, . . . , RN) is present in each branch circuit (21, . . . , 2N).

18. Ophthalmological laser treatment device according to claim 17, characterized in that, in each branch circuit (21, . . . , 2N) of the driver circuit (1), there is at least one switch (S1, SN) for connecting or disconnecting the series resistor (R1, . . . , RN).

19. Ophthalmological laser treatment device according to claim 17, characterized in that the driver circuit (1),
a) comprises series resistors (R1, . . . , RN) with the same resistance value and/or
b) comprises series resistors (R1, . . . , RN) with different resistance values, in particular in a series with doubled resistance values (RN=2^{(N-1)}R1).

20. Ophthalmological laser treatment device according to claim 17, characterized in that
a) in each branch circuit (21, . . . , 2N) of the driver circuit (1), the switch (S1, . . . , SN) is arranged in series with the series resistor (R1, . . . , RN) and/or
b) the switches (S1, . . . , SN) of the driver circuit (1) are MOSFET switches.

21. Ophthalmological laser treatment device according to claim 17, characterized in that the driver circuit (1) comprises digital electronics or a microcontroller (4) for controlling the switches (S1, . . . , SN), in particular in that the digital electronics or the microcontroller (4) is connected to each switch (S1, . . . , SN) via a respective actuation path (A1, . . . , AN).

22. Ophthalmological laser treatment device according to claim 21, characterized in that
a) a current sensor (8) for measuring the current through the laser or the laser diode (LD) is present in the circuit (2) of the driver circuit (1) and is connected to the digital electronics or the microcontroller (4) via a feedback path (80) for the current intensity control and/or
b) a light sensor (6, 7) is provided for the laser or the laser diode (LD) for measuring the light output (Pₒᵤₜ) of the laser or the laser diode (LD) and is connected to the digital electronics or the microcontroller (4) via a feedback path (70) for the light output control.

23. Ophthalmological laser treatment device according to claim 17, characterized in that
a) the voltage source (3) of the driver circuit (1) comprises means for varying its output voltage and/or
b) the laser or a laser diode (LD) is a laser diode-pumped or LED-pumped laser system, in particular a diode-pumped solid-state laser (DPSSL) or a coherently optically pumped semiconductor laser (OPSL) and/or
c) the driver circuit (1) comprises means for carrying out a method for operating the driver circuit (1), wherein in each branch circuit (21, . . . , 2N), a series resistor (R1, . . . , RN) and a switch (S1, SN) for connecting or disconnecting the series resistor (R1, . . . , RN) are present, characterized in that the series resistor is controlled or regulated in discrete stages by switching on or off at least one of the switches (S1, . . . , SN).
